(19) United States
(12) Patent Application Publication
(10) Pub. No.: US 2007/0141105 A1
(43) Pub. Date: Jun. 21, 2007

(54) Engineered Biological Matrices

(75) Inventors: Thomas Mark Stein, Myersville, MD (US); Mark James Powers, New Market, MD (US); Jonathan William Cooper, Walkersville, MD (US); Sorin Damian, Frederick, MD (US); Pieter Johannes Dirk Huiberts, Frederick, MD (US); Kenneth B. Guiseley, Hope, ME (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Cambrex Bio Science Walkersville, Inc.

(21) Appl. No.: 11/539,580

(22) Filed: Oct. 6, 2006

Related U.S. Application Data
(60) Provisional application No. 60/724,666, filed on Oct. 7, 2005.

(51) Int. Cl. A61F 2/02; A61K 9/14; C12N 5/02

(52) U.S. Cl. 424/423; 424/486; 435/404

(57) ABSTRACT

Biocompatible matrices or implants on which one or more specific cell-interactive molecules (“biomolecules”) can be immobilized have been developed. The matrices allow for the independent control of ligand concentration and matrix strength. In one embodiment, the matrix or implant is modified with one or more moieties capable of complexing bioconjugates prepared from one or more biomolecules. Suitable moieties include phenyl boronic acid complexing agents, such as salicylhydroxamic acid, which can complex to one or more biomolecules containing one or more phenyl boronic acid moieties. The biomolecules may be anchored to the matrix via a spacer molecule, which may allow for greater mobility of the biomolecules in aqueous solution. In one embodiment, the matrix is a hydrogel material which has been doubly-derivatized, wherein ligand concentration and matrix strength can be independently controlled. The matrices and implants can be used in vivo and in vitro applications including diagnostics, biosensors, bioprocess engineering, tissue engineering, regeneration and repair, and drug delivery.

### CROSS-REFERENCE TO RELATED APPLICATION

This application claims priority to U.S. Ser. No. 60/724,666, entitled “Engineered Biological Matrices”, filed Oct. 7, 2005.

### FIELD OF THE INVENTION

This invention is in the field of modified biocompatible matrices for use in tissue engineering, regeneration and repair or drug delivery.

### BACKGROUND OF THE INVENTION

Tissue engineering is generally defined as the creation of tissue or organ equivalents by seeding of cells onto or into a matrix suitable for implantation. The matrices must be biocompatible and cells must be able to attach and proliferate on the matrices in order for them to form tissue or organ equivalents. A number of different matrix materials have been utilized, including inorganic materials such as metals, natural polymeric materials such as fibrin and alginate, and synthetic polymeric materials such as polyhydroxyacids like poly(glycolic acid)(“PGA”) and copolymers thereof like poly(glycolic acid-co-lactic acid) (“PLGA”). Biodegradable polymeric materials are preferred in many cases since the matrix degrades over time and eventually the cell-matrix structure is replaced entirely by the cells.

Some matrix materials with desirable mechanical and processing characteristics do not demonstrate a high degree of cell attachment or proliferation. In some cases, it may be desirable to have different types of cells attach to different parts of a matrix; for example, a joint surface may include both bone and cartilage. A number of techniques have been used to enhance cell attachment, including linking bioactive molecules to the polymer forming the matrix, or simply coating the matrix material with another polymer having better cell attachment properties, although not the desired mechanical properties. In order to enhance attachment of specific cells to different regions of a matrix, multiple growth factors have been attached to distinct matrix areas—for example, fibroblast growth factor to enhance attachment and proliferation of chondrocytes to form cartilage, and bone morphogenic protein to enhance attachment and proliferation of bone-forming cells.

It is well known that the concentration of growth factors, cell adhesion molecules, and other bioactive agents is a major factor in cell attachment, proliferation and differentiation. This is particularly an issue when attaching pluripotent or multipotent cells to the matrix. Most techniques for coupling such bioactive agents require chemical modification of the polymers after formation of the matrix, which makes it extremely difficult to vary concentration of the active agents within the matrix. In the example of cell attachment, there exists a need for a cell matrix with defined cell adhesion molecules that can be synthesized and/or reconstituted to independently modify adhesion molecule concentration and/or matrix strength.

Therefore, it is an object of the present invention to provide matrices or implants that can be synthesized and/or reconstituted to independently modify bioactive molecule presentation—including biomolecule type, concentration, and spacing—and matrix mechanical properties.

It is another object of the present invention to provide matrices or implants which are modified with a complexing agent conjugated to a biomolecule and methods of making thereof.

It is still further an object of the present invention to provide matrices or implants that are modified with a complexing agent for use in tissue engineering, regeneration and/or repair or drug delivery.

### BRIEF SUMMARY OF THE INVENTION

Biocompatible matrices or implants on which one or more specific cell-interactive molecules (“biomolecules”) can be immobilized have been developed. The matrices allow for the independent control of both biomolecule concentration and matrix strength. In a preferred method of manufacture, the matrices are made using one or more different monomers or polymers having different densities of ligands thereon, which are mixed together to form all or part of a matrix having a defined ligand concentration, without altering the monomer or polymer concentration and/or matrix strength. In one embodiment, the matrix or implant is modified with one or more ligands capable of forming an affinity pair with a bioconjugate or other biomolecules. Suitable ligands include reactive sites such as aldehydes, epoxides, amines, activated carboxylic acids and vicinal diols. Other suitable ligands include one-half of the pair of binding partners such as streptavidin-biotin and phenyl boronic acid-salicylhydroxamic acid. Salicylhydroxamic acid can complex to one or more biomolecules containing one or more phenyl boronic acid moieties. Different types of ligands can be combined to allow binding of distinct groups of biomolecules. For example, an initial group of biomolecules could be bound to a matrix through one type of ligand followed by the binding of a second group of biomolecules to another type of ligand. The biomolecules may be anchored to the matrix via a spacer molecule that can allow for greater mobility of the biomolecules in aqueous solution. In one embodiment, the matrix is a hydrogel material which has been doubly-derivatized, wherein ligand concentration and gel strength can be independently controlled. The matrices and implants can be used in vivo and in vitro applications including diagnostics, biosensors, bioprocess engineering, tissue engineering, regeneration and repair, and drug delivery.

### BRIEF DESCRIPTION OF THE DRAWINGS

FIG. 1 is a schematic showing the reaction of a biomolecule with one molecule of a pair of binding partners to yield a bioconjugate. The lower half of the figure represents the other molecule of the pair of binding partners attached to the matrix capturing the prepared bioconjugate.

FIG. 2 is a schematic showing a matrix with covalently attached ligands capturing a bioconjugate from solution. This functionalized matrix is then employed to capture and immobilize cells to the matrix based on the choice of bioconjugate.

FIG. 3 is a schematic showing the ability to vary ligand spacing on the matrix while maintaining bulk ligand concentration and matrix strength. The total number of modified matrix sites is the same in both examples, but the localization of the sites with respect to each other is difficult.

FIG. 4 is a schematic showing the ability to vary matrix strength while maintaining functional ligand concentration and spacing. The total number of functional ligand sites and the spacing of the functional ligand modified sites are the same between both examples, while the total modification sites in the second example are higher. A higher number of synthetic modification sites yield a decrease in matrix strength independent of the type, size, or activity of the modification.

FIG. 5 is a schematic showing the ability to vary matrix strength and ligand spacing while maintaining ligand concentration. The total number of ligand sites is the same across both examples, while the example on the left demonstrates broader spacing across a higher number of polymer chains. A decrease in the number of total polymer chains per unit volume yields a decrease in the matrix strength.

FIG. 6 is a schematic showing the ability to maintain matrix strength while varying ligand concentration and spacing. By adding inert groups to the example on the right, the total number of modified sites remains the same while the number of functional ligand sites is lowered. A higher number of synthetic modification sites yield a decrease in matrix strength independent of the type, size, or activity of the modification. In this case, the matrix strengths are comparable as the number of modification sites is identical.

FIG. 7 is a schematic showing the ability to maintain matrix strength while varying the concentration of bioconjugate immobilized. Changes in matrix strength are related to the total number of modified sites and are independent of the type, size, or activity of the modification. The larger bioconjugates have little additional effect on matrix strength once the ligand concentration effect has been noted.

FIG. 8 is a schematic showing the ability to immobilize a mixture of multiple bioconjugates based on the use of a common ligand. The final ratio of the immobilized bioconjugates is determined by the initial ratio of bioconjugates in the mixture applied to the matrix.

FIG. 9 is a schematic showing the ability to either simultaneously (above) or sequentially (below) functionalize the matrix by employing multiple ligands and multiple bioconjugates on the matrix. The different ligands on the matrix are represented by the triangle and the X. The specific interaction between these ligands and the corresponding bioconjugates control the ratio and concentration of the bioconjugates immobilized on the matrix.

FIG. 10 is a schematic showing the ability to disrupt or block the ligand-bioconjugate interaction using a mimicking molecule that mimics the bioconjugate (example on the right) or the ligand (example on the left) effectively releasing the immobilized bioconjugates.

FIG. 11 is a schematic showing the ability to employ multivalent ligand and bioconjugate interactions to affect the avidity and matrix strength of the bioconjugate-matrix immobilization while maintaining ligand concentration.

Table 1 is a legend of the symbols used in these figures.

### DETAILED DESCRIPTION OF THE INVENTION

I. Definitions

“Biomolecules”, as used herein, refers to a biologically active agent such as proteins (including, but not limited to, cell adhesion proteins), growth factors, nucleic acids, synthetic polypeptides and inorganic and organic compounds, or complexes thereof. Biomolecules can also have features capable of complexing or reacting directly with a matrix surface.

“Bioconjugate”, as used herein, refers to a complex of two or more different molecular species coupled by chemical or biological means, in which at least one of the molecular species is a biomolecule and the other is a complexing agent. Bioconjugate can also refer to a biomolecule that has an inherent feature capable of complexing with the matrix surface.

“Ligand”, as used herein, refers to a coupling agent attached to the matrix and capable of binding a biomolecule or bioconjugate. For example, the ligand can be the matrix-bound binding partner of SHA-PBA or avidin-biotin complexes. It can also be a reactive group which can directly couple a biomolecule to the matrix surface by covalent or noncovalent interaction.

“Complexing agent”, as used herein, refers to the target of the ligand immobilized on the matrix. For example, the complexing agent can be the binding partner of SHA-PBA or avidin-biotin complexes that is conjugated to the biomolecule. It can also be the feature of a biomolecule targeted by the ligand in direct coupling.

“Inert groups”, as used herein, refers to modification of the matrix that modulate ligand concentration or physical properties such as matrix strength. These groups are generally inert in their ability to complex bioconjugates or interact with cells.

“Matrix” or “matrices”, as used herein, refers to the substance or substances to which ligands are bound for the immobilization (complexation) and presentation of biomolecules and bioconjugates. Matrix materials include, but not limited to, solid surfaces or gel networks such as hydrogels. In the case of gel networks, the physical properties of the gel can be tailored to the desired application.

“Ligand concentration”, as used herein, refers to the absolute concentration of the ligand, whether in the context of the degree of substitution of a ligand within a solvent-free polymer, or within a volume of solution or a hydrogel formed from a ligand-bearing polymer. Ligand concentration can also refer to the absolute concentration of a ligand on a two-dimensional surface.

“Ligand spacing”, as used herein, refers to the relative distance between ligand groups, whether in a linear sense as they are distributed among a linear polymer, in a two-dimensional sense as they are distributed on a solid surface or the cell-accessible surface of a hydrogel, or in a three-dimensional sense as they are distributed within the volume of a solution or hydrogel. When ligand spacing is small, even when overall ligand concentration is low, the ligands can be considered clustered together. Ligand clustering is relevant in certain biological functions, such as cell adhesion. Where ligand spacing is great, ligands can be considered diffuse.

“Matrix concentration”, as used herein, refers to the absolute concentration of matrix materials including, but not limited to, the polymeric components within the volume of a hydrogel.

“Biocompatible”, as used herein, refers to materials that do not produce a toxic, injurious or immunological response in living tissue.

“Biodegradable”, as used herein, refers to materials that degrade in vivo to non-toxic compounds, which can be excreted or further metabolized.

“Phenyl boronic acid” (“PBA”), as used herein, refers to a molecule containing one or more phenyl boronic acid groups. The phenyl boronic acid species can comprise one, two, or three boronic acid groups attached at various positions about the aromatic drug.

“Salicylhydroxamic acid” (“SHA”), as used herein, refers to a molecule that contains one or more groups able to form a complex with another molecule containing one or more phenyl boronic acid groups.

“Hydrogel”, as used herein, refers to polymers that swell extensively in water but are not water soluble. “Organogel”, as used herein, refers to a material formed by mixing small amounts of an organic molecule in a liquid solvent in which the organic molecules spontaneously aggregate trapping solvent molecules.

“Elastomeric”, as used herein, refers to a flexible, low modulus material capable of expanding and contracting and returning to its original dimensions without fatigue.

Composition

Biocompatible matrices on which a specific biomolecule or combination of biomolecules can be immobilized have been developed. The immobilization technique involves the affinity interaction of selecting binding partners including, but not limited to, phenylboronic acid with salicylhydroxamic acid or streptavidin with biotin, which are covalently attached to the biomolecules and the matrix. The matrices allow for the independent control of ligand concentration and matrix strength.

Naturally occurring cell matrices, such as collagen and Matrigel™, typically comprise proteins that serve both as active cell binding substrates as well as structured supports. Because of this, key molecular and physical properties of the matrices, such as biomolecule concentration and matrix strength, cannot be decoupled or independently varied. Furthermore, these matrices typically cannot incorporate other cell-interactive molecules, such as cytokines, in a controlled manner.

In order to address the limitations associated with naturally occurring matrices, synthetic matrices, often made from hydrogels, were developed. Modification of the biomolecule concentration in reconstituted hydrogel matrices, however, requires changing the matrix concentration, which in turn alters the hydrogel strength. Exogenous mechanisms to increase or decrease gel strength, such as derivatization of the matrix with glutaraldehyde, have been developed, but such procedures introduce modifications into the system.

Methods and materials have been developed which solve these problems associated with the prior art materials. One or more materials are derivatized with ligands capable of binding bioconjugates. In the case of hydrogels consisting of polymeric component molecules, ligand concentration can be controlled through the number of ligand sites per polymer chain, or through the concentration of ligand-bearing polymer chains within the matrix. Matrix strength is controlled by numerous factors, including polymer concentration, the presence of disruptive inert ligands, polymer chain length, and degree of polymer chain crosslinking. Control of these parameters can be combined to target a matrix with the desired mechanical properties, ligand concentration and ligand spacing.

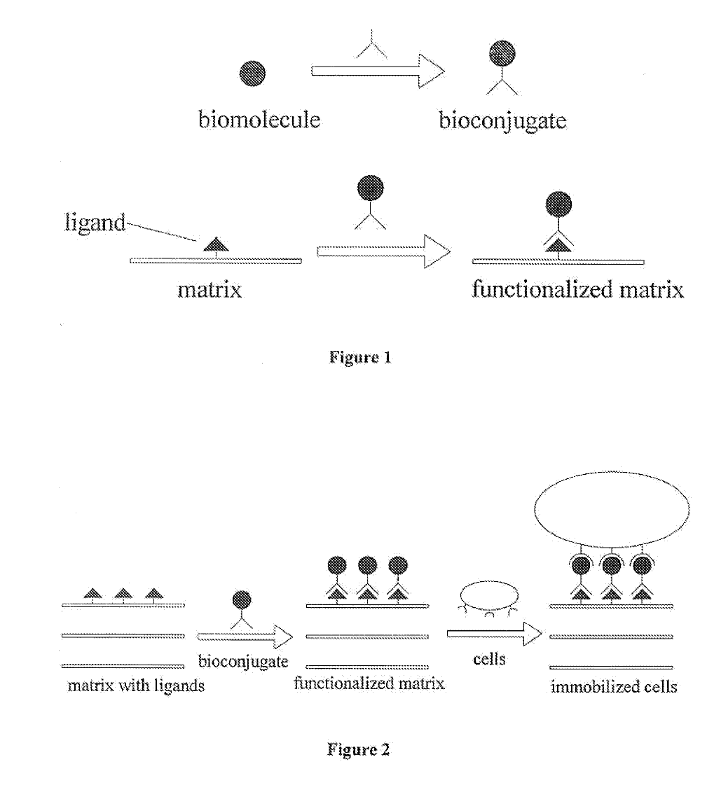

FIGS. 1-11 demonstrate various embodiments of the compositions and how attachment of ligands may be manipulated to vary the properties of the matrix. Table 1 is the legend for these figures.

TABLE 1
Legend for Figures

| | |
|---|---|
| [symbol] | biomolecule |
| [symbol] | PBA |
| [symbol] | SHA |
| [symbol] | biotin |
| [symbol] | avidin |
| [symbol] | bioconjugates |
| [symbol] | SHA mimic |
| [symbol] | PBA mimic |
| [symbol] | inert group |
| [symbol] | multivalent PBA |
| [symbol] | multivalent SHA |
| [symbol] | 3x % matxix hydrogel |
| [symbol] | x % matrix hydrogel |
| [symbol] | cell |
| [symbol] | cell-surface receptor |

Ligands on the matrix are used to couple biomolecules to create defined functionalized, biologically active matrices (FIG. 1). Such tailored matrices can then be used to immobilize and control the biological function of cells (FIG. 2).

For example, in one embodiment, ligand spacing can be controlled by derivatizing a matrix polymer, then blending this derivatized polymer with a second, larger fraction of underivatized polymer to yield a clustered ligand spacing. In another embodiment, the matrix can be composed entirely of a polymer with a lower degree of derivatization to yield a diffuse ligand spacing.

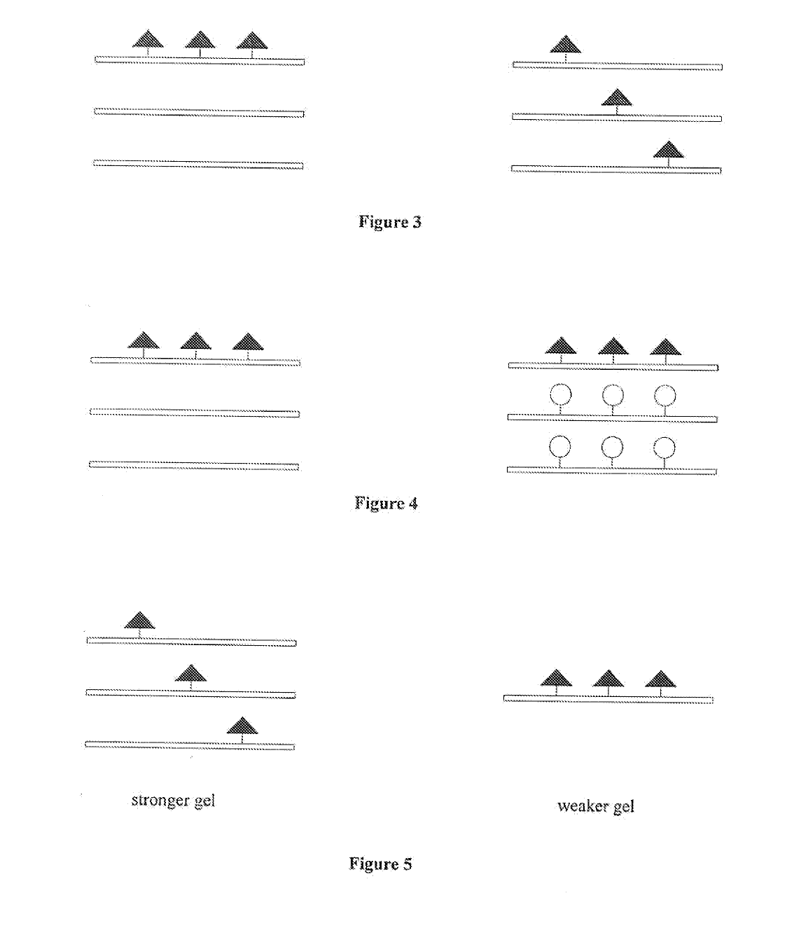

As shown in FIG. 3, by judicious selection of ligand derivatization levels and polymer blending ratios, matrices can be formed with any desired ligand spacing between these two extremes while holding bulk matrix ligand concentration constant. Likewise, overall polymer concentration can be maintained at the same level between these two embodiments to ensure a consistent matrix strength.

In a third embodiment shown in FIG. 4, a ligand-bearing polymer is blended with a larger fraction of underivatized matrix polymer. By introducing inert disruptive groups in the ligand-free polymer (open circles in FIG. 4) while maintaining the ratio between ligand-bearing polymer and ligand-free polymer, a weaker matrix can be formed at the same ligand spacing and concentration. By judicious selection of the concentration and type of inert disruptive group on the ligand-free polymer, matrices with any desired strength can be formed between these two extremes, while maintaining a consistent ligand spacing and concentration.

In a fourth embodiment shown in FIG. 5, matrix strength and ligand spacing can be modified at a constant bulk ligand concentration. In this case, a matrix such as a hydrogel is prepared from a polymer that contains a given ligand concentration. A second matrix is prepared using a polymer with a higher level of ligand derivatization. By maintaining the relative polymer concentration at levels that are inversely proportional to the relative degree of ligand derivatization, ligand concentration is held constant. The more highly derivatized polymer composes a weaker matrix with clustered ligand spacing relative to the stronger, more diffuse ligand-containing matrix comprising the less derivatized polymer. By judicious selection of derivatization level and matrix concentration, hydrogels with any desired gel strength between these two extremes can be prepared, while maintaining a consistent ligand concentration.

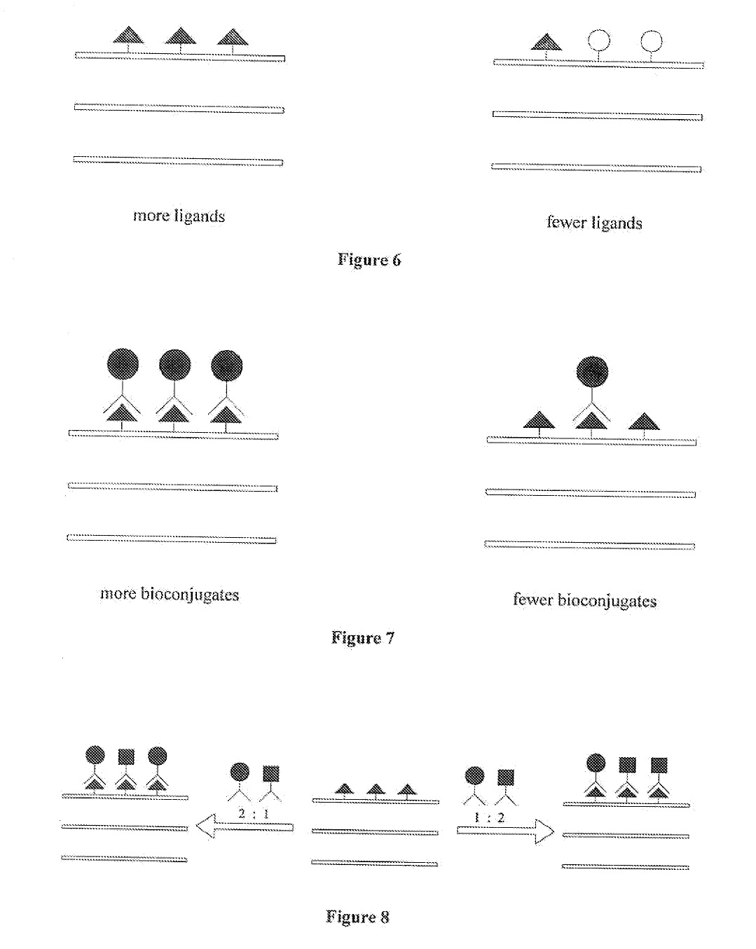

In a fifth embodiment shown in FIG. 6, ligand spacing on one fraction of the matrix polymer is controlled through the use of inert groups (open circles in FIG. 6) that occupy a portion of the reactive sites on the polymer. By judicious selection of ratios of ligand and inert groups during the derivatization step, a single reactive, fully-derivatized polymer can be used to create any desired ligand content between the extremes of a completely ligand-bearing polymer to a completely inert-group-bearing polymer. These ligand-bearing polymers can subsequently be blended with underivatized polymer to prepare hydrogels with any desired ligand concentration and spacing, while maintaining a consistent matrix strength.

In a sixth embodiment shown in FIG. 7, the concentration of bioconjugates immobilized on the matrix is controlled by the concentration or time that the matrix is exposed to the bioconjugate during the functionalization step. The concentration of the ligand on the matrix places an upper limit on the maximum concentration of immobilized bioconjugates. Exposing the matrix to a greater-than-stoichiometric amount of bioconjugates for an extended period of time will produce a matrix with maximum loading with respect to the available ligands. The use of less-than-stoichiometric amounts of bioconjugates and/or relatively short exposure times will produce a matrix with less than the maximum loading, with respect to the available ligands. Given that the ligand itself has an insignificant biological response, the interaction between a cell and the matrix will be controlled solely by the concentration of the biomolecule. By judicious selection of bioconjugate concentration and exposure time during matrix functionalization, a single matrix material can be used to prepare functionalized matrices within a range of bioconjugate concentrations to control biological response, while maintaining a consistent matrix strength.

In a seventh embodiment shown in FIG. 8, the composition of the functionalized matrix is controlled by the relative ratios of bioconjugates used during the functionalization step. By using a mixture of bioconjugates, where the biomolecules share a common complexing agent, matrices can be prepared with defined ratios of two or more immobilized biomolecules.

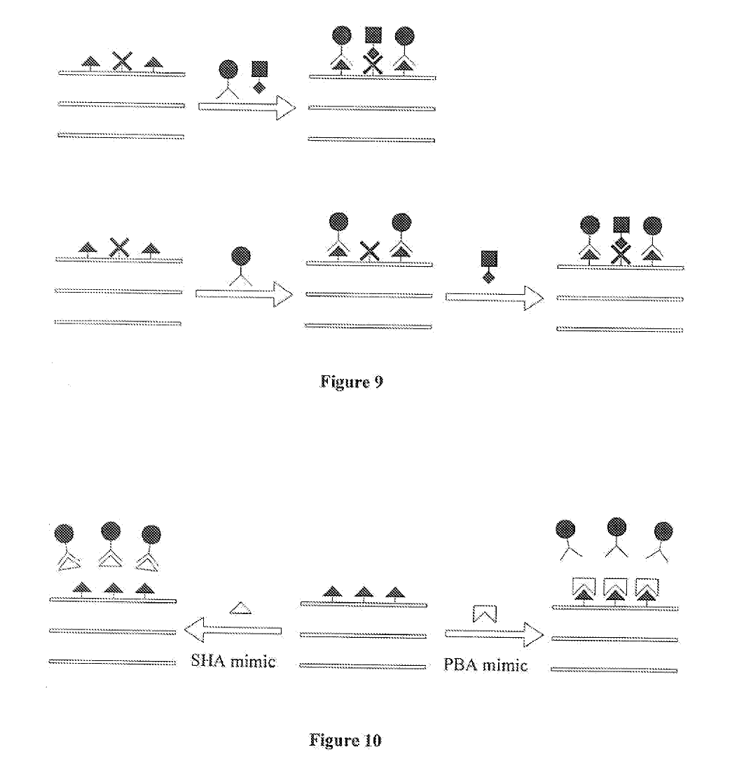

In an eighth embodiment shown in FIG. 9, the composition of the functionalized matrix is controlled by using bioconjugates with different biomolecules attached to different complexing agents. Providing that the ligand interactions are specific for particular complexing agents, biomolecule mixtures can be immobilized in a controlled manner to prepare a matrix with a defined mixture of biomolecules. Specific ligand-complexing agent interactions allow the functionalization step to be done either simultaneously with a bioconjugate mixture or, when necessary, sequentially with separate mixtures of bioconjugates.

In a ninth embodiment shown in FIG. 10, the interaction of cells or other biological entities with the functionalized matrix can be controlled through exposure to molecules that compete with the ligand or complexing agent for binding, or otherwise disrupt the complexation of the bioconjugates to the matrix surface.

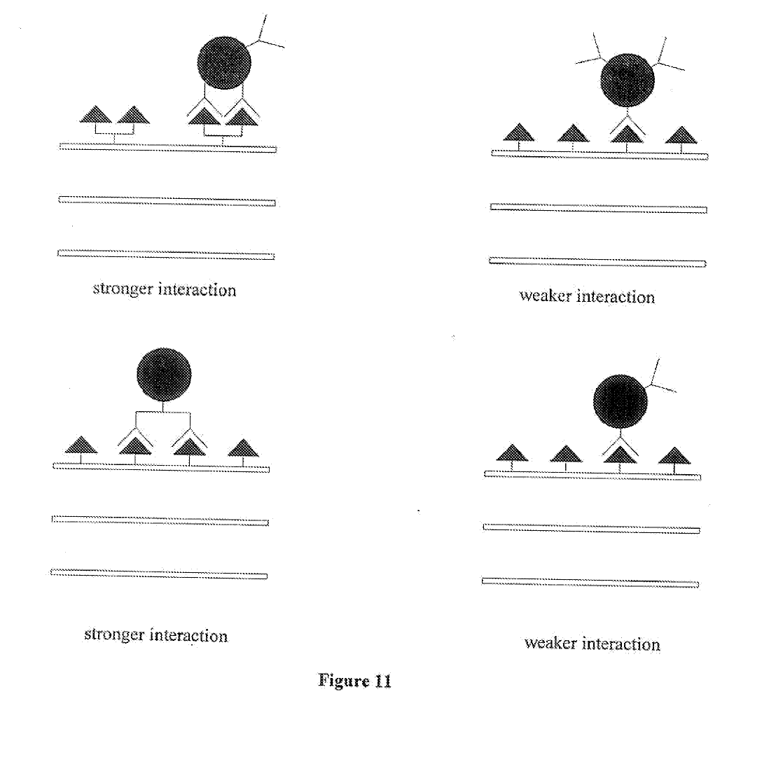

In a tenth embodiment shown in FIG. 11, the strength of complexation between the bioconjugate and the matrix is controlled through the use of monovalent or multivalent ligand or complexing agent molecules. Multivalent molecules generally lead to stronger, more stable interactions than an equivalent concentration of their monovalent counterparts. There may be circumstances where a weaker interaction is more favorable, for example, in cases where release of the bioconjugate is desired. The ability to introduce two immobilization points from one modification site is useful in the control of ligand spacing, ligand concentration, and ultimately matrix strength. Additionally, an increase in the number of ligand-bioconjugate interactions increases the strength of the immobilization.

The foregoing embodiments illustrate the range of control afforded by the invention over biomolecules on functional matrices. These methods of control can also be combined to afford an even greater range of control.

A. Coupling or Complexing Agents
i. Direct Chemical Coupling Agents

Biomolecules can be conjugated and immobilized on a matrix using a wide variety of ligands including, but not limited to, aldehydes by reductive amination, epoxides and activated esters by nucleophilic attack, and amines in cases where the biomolecule contains one or more electrophilic sites including, but not limited to, activated esters. Reactive sites can be generated in situ, for example, via the reaction of vicinal diols with periodate to form reactive aldehydes. If only a portion of the diols is converted to aldehydes, a double derivative composed of inert diols and reactive aldehyde groups is formed, as represented in FIG. 6. A double derivative can also be formed by reacting the available aldehydes with a mixture of active and inert reagents. In the case of an agarose matrix, the presence of inert and reactive groups can be used to modulate the physical properties of the matrix.
ii. Streptavidin-Biotin Coupling

Streptavidin or avidin is a tetrameric protein that binds tightly to the small molecule biotin to form strong, stable and specific complexes. Each monomer of streptavidin binds one molecule of biotin. Biotin is a water-soluble vitamin, generally classified as a B-complex vitamin. The structure of biotin is shown below:

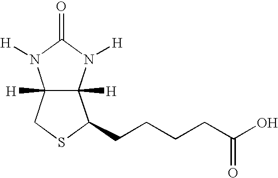

In one embodiment, streptavidin can be the ligand bound to the matrix. The tetrameric nature of streptavidin can produce a multiplying effect by binding up to four biotin-conjugated biomolecules.
iii. Polyhistidine-Nickel Chelate Coupling

Stable complexes can be formed by reacting polyhistidine tags with chelated nickel cations including, but not limited to, Ni²⁺ tridentate or Ni²⁺ nitrilotriacetic acid. In one embodiment, the matrix can be derivatized with a polyhistidine tag ligand which can form a complex with a Ni²⁺ tridentate or nitrilotriacetic-derivatized biomolecule.
iv. Salicylcylhydroxamic Acids

Reagents suitable for the modification of the matrix material for the purpose of attaching a salicylhydroxamic acid moiety for subsequent conjugation/complexation to one or more biomolecules having pendant phenyl boronic acid groups have the general formula shown below: 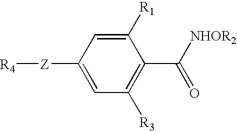
wherein R₄ is a reactive electrophilic or nucleophilic moiety suitable for reaction of the salicylhydroxamic acid molecule with the matrix material or R₄ is a moiety capable of reacting in a redox process, e.g., the formation of a disulfide bond. R₂ is an H, an alkyl, or a methylene or ethylene moiety with an electronegative substituent. R₁ and R₃ are independently H or hydroxy and Z is optionally a spacer molecule comprising a saturated or unsaturated chain from 0 to 6 carbon equivalents in length, an unbranched or branched, saturated or unsaturated chain from 6 to 18 carbon equivalents in length with at least one intermediate amine or disulfide moiety, or a polyethylene glycol chain of 3-12 carbon equivalents in length. In one embodiment, the salicylhydroxamic acid ligand is attached to the surface through the agent salicylhydroxylamine hydrazide. In other embodiments, the salicylhydroxamic acid ligand can be attached to the surface with a salicylhydroxylamine N-hydroxysuccinimide (“NHS”) ester or carboxylic acid.
v. Phenyl Boronic Acids

Phenyl boronic acid reagents, many of which are known in the art, can be appended to a biomolecule to afford a conjugate having one or more pendant phenyl boronic acid moieties as shown below:

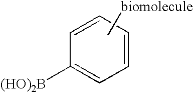

The reagent may include a group comprising a spacer molecule such as an aliphatic chain up to 6 carbon equivalents in length, an unbranched aliphatic chain of 6 to 18 carbon equivalents in length with at least one intermediate amide or disulfide moiety, or a polyethylene oxide or polyethylene glycol chain of 3-12 carbon equivalents in length. The use of spacer molecules such as polyethylene oxide and polyethylene glycol may allow for higher mobility of the biomolecule/bioconjugate in aqueous solution. The biomolecule may also include a portion of a reactive moiety used to attach the biomolecule to the phenyl boronic acid species in the absence of a spacer molecule. The phenyl boronic acid species can comprise one, two, or three boronic acid groups attached in various positions about the aromatic ring.

B. Linkers

Linkers can be used between the matrix and ligands such as phenyl boronic acid or salicylhydroxamic acid, or between the biomolecule to be bound to the matrix and the phenyl boronic acid or salicylhydroxamic acid. For example, flexible linkers, or “tethers”, may be used for attaching growth factor molecules to a substrate. Substantial mobility of a tethered growth factor is critical because even though the cell does not need to internalize the complex formed between the receptor and the growth factor, it is believed that several complexes must cluster together on the surface of the cell in order for the growth factor to stimulate cell growth. In order to allow this clustering to occur, the growth factors are attached to the solid surface, for example, via long water-soluble polymer chains, allowing movement of the receptor-ligand complex in the cell membrane.

Examples of water-soluble, biocompatible-polymers which can serve as tethers include, but are not limited to polymers such polyethylene oxide (PEO), polyvinyl alcohol, polyhydroxyethyl methacrylate, polyacrylamide, and natural polymers such as hyaluronic acid, chondroitin sulfate, carboxymethylcellulose, and starch.

Tethers can also be branched to allow attachment of multiple molecules in close proximity. Branched tethers can be used, for example, to increase the concentration of growth effector molecule on the substrate. Such tethers are also useful in bringing multiple or different growth effector molecules into close proximity on the cell surface. This is useful when using a combination of different growth effector molecules. Preferred forms of branched tethers are star PEO and comb PEO.

Star PEO is formed of many PEO “arms” emanating from a common core. Star PEO has been synthesized, for example, by living anionic polymerization using divinylbenzene (DVB) cores, as described by Gnanou et al., Makromol. Chemie 189: 2885-2892 (1988), and Merrill, J. Biomater. Sci. Polymer Edn 5: 1-11 (1993). The resulting molecules have 10 to 200 arms, each with a molecular weight of 3,000 to 12,000. These molecules are about 97% PEO and 3% DVB by weight. Other core materials and methods may be used to synthesize star PEO. Comb PEO is formed of many PEO chains attached to and extending from the backbone of another polymer, such as polyvinyl alcohol. Star and comb polymers have the useful feature of grouping together many chains of PEO in close proximity to each other.

It is desirable for tether length and strength to be matched to give a desired half-life to the tether, prior to breakage, and thereby adjust the half-life of the bound molecule or its effect, for example, growth factor action. The minimum tether length also depends on the nature of the tether. A more flexible tether will function well even if the tether length is relatively short, while a stiffer tether may need to be longer to allow effective contact between a cell and the growth effector molecules.

The backbone length of a tether refers to the number of atoms in a continuous covalent chain from the attachment point on the substrate to the attachment point of the molecule. All of the tethers attached to a given substrate need not have the same backbone length. In fact, using tethers with different backbone lengths on the same substrate can make the resulting composition more effective and more versatile. In the case of branched tethers, there can be multiple backbone lengths depending on where and how many molecules are attached. Preferably, tethers can have any backbone length between 5 and 50,000 atoms. Within this preferred range, it is contemplated that backbone length ranges with different lower limits, such as 10, 15, 25, 30, 50, and 100, will have useful characteristics.

Biocompatible polymers and spacer molecules are well known in the art and most are expected to be suitable for forming tethers. The only important characteristics are biocompatibility and flexibility. That is, the tether should not be made of a substance that is cytotoxic or, in the case of in vivo uses, which causes significant allergic or other physiological reaction when implanted.

One of the advantages of these conjugates is that, unlike typical cell culture methods that require the use of trypsin or other enzymatic materials to proteolytically degrade the cell/matrix attachment, which damages the cells and disrupts functionality, release of the entire receptor-ligand complex from the surface provides for a benign release of the cells from the surface for evaluation or further subculture.

C. Matrix Materials

The matrix or implant may be formed from rigid, elastomeric or gel-like materials (hydrogels or organogels). The matrix or implant can be formulated in order to vary the physical and mechanical properties such as biomolecule concentration, biomolecule distribution, tensile strength, etc. in order to meet the requirements of different cell types. The matrix or implant can be used for both in vitro or in vivo applications.

There are two basic types of substrates: biocompatible materials which are not biodegradable including, but not limited to, polystyrenes, polyethylene vinyl acetates, polypropylenes, polymethacrylates, polyacrylates, polyethylenes, polyethylene oxides, glass, polysilicates, polycarbonates, polytetrafluoroethylene, fluorocarbons, nylon, silicon rubber, and stainless steel alloys, and titanium alloys; and biocompatible, biodegradable materials including, but not limited to, polyanhydrides, polyglycolic acid, polyhydroxy acids such as polylactic acid, polyglycolic acid, and polylactic acid-glycolic acid copolymers, polyorthoesters, polyhydroxyalkanoates, polyphosphazenes, polypropylfumerate, biodegradable polyurethanes, proteins such as collagen, polyamino acids, polysaccharides such as glycosaminoglycans, alginate, agarose, and carageenan, bone powder or hydroxyapatite, and combinations thereof. These biodegradable polymers are preferred for in vivo tissue growth scaffolds. Other degradable polymers are described by Engleberg and Kohn, Biomaterials 12: 292-304 (1991). For implantation in the body, preferred degradation times are typically less than one year, more typically in the range of weeks to months.

Attachment substrates can have any useful form including substrates for cell culture such as bottles, dishes, fabrics and fibers such as sutures, woven fibers, and non-woven fabrics, implants such as shaped polymers, particles and microparticles, bone cements, and temporary implants such as stents, coatings, and catheters. For in vitro cell growth, the growth effector molecules can be tethered to standard tissue culture polystyrene Petri dishes. Woven fibers are useful for stimulating growth of tissue in the form of a sheet, sponge or membrane. In general, matrices for tissue repair or regeneration will be porous or fibrous structures having pore diameters or interstitial spacing of at least 100 microns if the matrix is to be seeded with cells and cultured initially in vitro. Pores can be created by inclusion of water-soluble or volatile salts at the time the polymer solution is cast or molded, then removed by solvent leaching or evaporation.

In some embodiments, attachment of the cells to the substrates is enhanced by coating the substrate with compounds such as extracellular membrane components, basement membrane components, agar, agarose, gelatin, gum arabic, collagen types I, II, III, IV, and V, fibronectin, laminin, glycosaminoglycans, mixtures thereof, and other materials known to those skilled in the art of cell culture.

In one embodiment, the matrix or implant is a hydrogel, defined as a substance formed when an organic polymer (natural or synthetic) is crosslinked via covalent, ionic, or hydrogen bonds to create a three-dimensional open-lattice structure that entraps water molecules to form a gel. Examples of materials which can be used to form a hydrogel include, but are not limited to, natural polymers including polysaccharides such as alginate, hyaluronic acid, and agarose and proteins such as fibrin and collagen, as well as synthetic polymers like polyphosphazines, and polyacrylates, which are crosslinked ionically, or block copolymers such as Pluronics™ or Tetronics™, which are polyethylene oxide-polypropylene glycol block copolymers which are crosslinked by temperature or pH, respectively, or polymers such as polyvinylpyrrolidone. Derivatization of the hydrogel with the ligand can occur before or after gelation.

Agarose is a long, linear polysaccharide composed of a basic repeating unit containing D-galactose and 3,6-anhydro-L-galactose. Like its source material agar, agarose forms firm gels at low concentrations in the range of 1% when dissolved in hot water and then cooled. These gels can be remelted by heating. During gelation, agarose forms double helices that further assemble to form helical bundles. The bundles, and the chains of agarose that connect them, form the large pores, typically 200 nm, characteristic of agarose gels. Unlike agar, agarose is substantially uncharged and biomolecules have an inherently low affinity for the neutral agarose molecule, making it an ideal basis for functional derivatives. A large number of agarose derivatives are commercially available. These are commonly in the form of crosslinked agarose beads used for biomolecular separations. Derivatives include charged groups that allow agarose to act as an ion exchange medium in which biomolecules are captured through general charge interactions. Derivatizing groups can also be proteins, with very specific biomolecule capture mechanisms, for example, through antibody-antigen interactions. The strong affinity of the avidin-biotin interaction also forms a useful line of agarose derivatives. The range of derivatized linear agarose is narrower than that of crosslinked agarose. Rather than emphasizing biomolecular interaction, derivatization of linear agarose is usually directed at modifying its gelling and melting properties.

The effect of derivatizing groups synthetically added to agarose is substantially different from those groups added by nature. As the degree of substitution (“DS”) by synthetic methods increases, the gelling temperature of agarose drops. Substitution by natural processes has the opposite effect. This difference in the results between substitution methods is related to which particular sites on the repeating disaccharide unit are substituted. By synthetic methods, it appears the most favorable reaction site causes the derivatizing group to interfere with the gelation mechanism. The size of the group has little effect on the reduction of the gelling temperature. Synthetic derivatization lowers both the gelling temperature and the strength of the agarose gel. While gelling temperature is moderately dependent on agarose concentration, gel strength is strongly dependent on concentration. By using the relatively independent controls of degree of substitution and concentration, it is possible to tailor the physical properties of agarose gels. This is applicable to some of the other materials as well.

While crosslinking allows agarose gels to be derivatized to a very high degree of substitution, it also destroys the gels thermoreversible character. Crosslinked gels cannot ordinarily be remelted. Linear agarose is typically derivatized with benign groups (methyl, hydroxyethyl, etc.) to control its gelling and melting properties, but excessive derivatization can abolish its ability to gel. Therefore, there is a maximum limit of substitution, above which the agarose derivative can no longer gel. Since it is the presence of the derivatizing groups, rather than their size, that determines the swelling characteristics of an agarose gel, one can further react some or all of these groups with bioactive molecules without dramatically altering the properties of the gel. Within the maximum limit of substitution, one can independently control the concentration of liquid (and, in turn, biomolecules) and physical properties of the agarose derivative gel. This forms the basis of tailored agarose matrices for cell matrices using a double-derivative approach.

Beyond the controls of overall DS, the percentage of bioactive substituents, and agarose concentration, one can independently vary the concentration of the substituents on particular agarose molecules through blending of derivatives. The physical characteristics of the agarose molecules will be largely determined by the greatest fraction of derivative. For example, if a lightly derivatized agarose, which has retained much of its gelling and strength characteristics, is blended with a smaller fraction of highly derivatized agarose containing bioactive groups, the resulting gel will have physical properties more closely associated with the lightly derivatized agarose. The resulting gel will have strength as well as areas of concentrated bioactive groups (ligand). This forms the basis of tailored agarose matrices for cell matrices using a dilution approach.

The matrix can also be used to control multiple ligand densities by derivatizing the matrix with different types of ligands. The different ligand densities can be controlled through stoichiometry since the on-off rates are dependent on the specific linking reagents.

Agarose can be degraded via an agarose enzyme. This does not affect cell-matrix interactions, but degrades the agarose backbone. This differs from other enzymatic approaches, which attack the proteins of the three dimensional matrices.

D. Bioactive Molecules

Therapeutic, prophylactic and diagnostic agents can be incorporated into the matrix for delivery, or attached on or to the matrix to enhance cell attachment and/or growth. Examples of suitable therapeutic agents include, but are not limited to, proteins, such as hormones, antigens, and growth effector molecules; nucleic acids, such as antisense molecules; and small organic or inorganic molecules such as antibiotics, steroids, decongestants, neuroactive agents, anesthetics, and sedatives. Examples of suitable diagnostic agents include radioactive isotopes, readiopaque agents and magnetic compounds. The compositions can include more than one active agent.

Growth effector molecules, as used herein, refer to molecules that interact with cell surface receptors and regulate the adhesion, growth, replication, or differentiation of target cells or tissue. Preferred growth effector molecules are growth factors and extracellular matrix molecules. Examples of growth factors include, but are not limited to, epidermal growth factor (EGF), platelet-derived growth factor (PDGF), transforming growth factors (TGF-α, TGF-β), hepatocyte growth factor, heparin binding factor, insulin-like growth factor I or II, fibroblast growth factor, erythropoietin, nerve growth factor, bone morphogenic proteins, muscle morphogenic proteins, and other factors known to those of skill in the art. Additional growth factors are described in “Peptide Growth Factors and Their Receptors I” M. B. Sporn and A. B. Roberts, eds. (Springer-Verlag, New York, 1990), for example.

Examples of extracellular matrix molecules include, but are not limited to, fibronectin, laminin, collagens, and proteoglycans. Other extracellular matrix molecules are known to those skilled in the art. Other growth effector molecules useful for tethering include cytokines, such as the interleukins and GM-colony stimulating factor, and hormones, such as insulin. These are also described in the literature and are commercially available.

Ligands for specific cell types may be attached to matrices to facilitate selective cell attachment.

III. Method of Making

One of the benefits of this system is that one can independently control matrix mechanical strength and/or growth effector molecule concentration. Naturally occurring cell matrices such as collagen and Matrigel™ have fixed numbers of cell adhesion domain densities per individual component molecule. Modification of cell adhesion domain concentration in a reconstituted hydrogel matrix therefore requires changing the matrix concentration, which alters matrix strength; so it is not possible to independently modify cell adhesion domain concentration and matrix mechanical strength without using exogenous means. Exogenous mechanisms to increase or decrease gel strength such as glutaraldehyde introduce modifications to the system. The materials described herein have the benefit of being independently modifiable so that defined ligand binding sites can be synthesized and/or reconstituted to independently modify biomolecular concentration or matrix strength.

Salicylhydroxamine Acid (SHA) Derivatized Matrices

In one embodiment, independent control of ligand concentration and matrix strength is achieved using a double-derivative method. Derivatized agarose chains are synthesized with various stoichiometric ratios of SHA-to-inert sites. For example, both SHA-X-hydrazide and acetic hydrazide are similarly reactive towards agarose chains derivatized to contain aldehyde groups. Only sites where aldehyde groups react with SHA-X-hydrazide would be active toward PBA-derivatized biomolecules. The remaining sites, where aldehyde groups react with acetic hydrazide, would be considered inert. This approach allows for a pre-determined modification of ligand concentration at constant gel strength. This is shown schematically in FIG. 6.

B. Phenyl Boronic Acid (PBA) Derivatized Biomolecules

To derivatize a biomolecule with PBA, several group-specific reagents are available, which target, for example, amines, aldehydes, thiols and activated carboxyl groups. Following reaction with the appropriate PBA reagent, the reaction mixture can be purified of unwanted by-products and reactants by either passing the mixture through a 500 MWCO size exclusion column (such as Sephadex®) or by dialysis in the desired buffer solution. Under certain circumstances, removal of unreacted reagents is unnecessary and the derivatized biomolecule can be used directly.

PBA-derivatized Biomolecule Immobilization on SHA-derivatized Matrices

The bioconjugate beween the PBA-derivatized biomolecules and the matrix derivatized with SHA can be formed through a variety of procedures known in the literature.

The PBA-SHA interaction can be disrupted using various molecular release approaches, such as SHA mimics or PBA mimics, as shown in FIG. 10. This approach has been used for various protein binding applications, but not cell culture applications. Such approaches can be used to effect the release of cells or molecules of interest from a functionalized matrix.

This interaction can also be controlled by modification of the avidity of the interaction (FIG. 11). In the case of a cell adhesion biomolecule, this approach allows for cell adhesion to the surface to be modified from weak/reversible to strong/irreversible. It could also allow for growth factor biomolecular release from the matrix to a soluble firm in the medium that can be readily internalized by cells.

IV. Methods of Use

The matrices and implants are formed as described above wherein the composition, type, and concentration of the binding receptor/ligand are determined by the ultimate use. For example, for tissue engineering, the material may be a fiber for use as a suture or woven or non-woven fabric which can be seeded with cells, where selective cell attachment or growth is a function of the bound molecules; a system for the screening of therapeutic or toxic materials, where the cells are bound in different regions or channels within the matrix and the device is perfused; liquids or suspensions which are solidified in situ for subsequent attachment, proliferation or in growth of cells especially in the case of bone where bone morphogenic protein is attached to the matrix; or substrates such as a plastic, glass/silicone or metal that are used as an implant and the receptor ligand is critical to promote adequate attachment.

The materials can also be used for drug delivery or diagnostic use. Release and dosage will be determined by selection of the ligands and molecules to be bound thereto, as described above.

These materials are utilized in vitro or in vivo as appropriate for the material, using the methods and materials known to those skilled in the art of cell culture and tissue engineering.

Materials and Methods:

### Example 1

### Preparation of Agarose Matrices with Similar Ligand Concentration and Varying Gel Strength

A series of agarose samples (A-E) were prepared with identical ligand concentration and varying gel strength. A derivatized agarose concentrate was prepared by suspending 10 g NuFix® Clyoxal Agarose with a binding capacity of 0.280 meq/g (Cambrex Bio Science) in a 400 mL aqueous solution of 2.5 mM SHA-X-hydrazide (Cambrex Bio Science) and 8 mM acetic hydrazide (Sigma-Aldrich). The 1:3.2 ratio of SHA-X-hydrazide to acetic hydrazide was assumed to be reflected in the corresponding immobilized groups. After 1 hour, the liquid was separated from the derivatized gel.

The moist, derivatized gel was then divided into 5 equal parts of equal mass and each portion suspended in 200 mL water. To each suspension was added one 6 g portion of five different agarose powders (SeaKem® Gold, SeaKem® LE, SeaKem® HGT, HSB-LV, and SeaPlaque®, all from Cambrex Bio Science). The underivatized agarose materials had gel strengths ranging from >200 g/cm2 (1% SeaPlaque®, weakest) to >1,800 g/cm2 (1% SeaKem® Gold, strongest).

Each of the separate agarose suspensions was heated to boiling to dissolve the agarose. After cooling, the gels were cut into approximately 5 mm cubes, which were frozen and thawed, before drying in a convection oven. The resulting flakes were ground to pass a 1 mm mesh to yield SHA agarose derivatives as free flowing powders with identical SHA ligand concentration.

### Example 2

### Preparation of Agarose Matrices with Varying Clustered Ligand Concentration and Similar Gel Strength

A pair of agarose samples (F and G) was prepared with varying ligand concentration and similar gel strengths. A derivatized agarose concentrate was prepared by the approach described above, but altering the ratios of the SHA-derivatized NuFix® and the SeaKem® LE to provide two levels of ligand concentration.

A derivatized agarose concentrate was prepared by suspending 165 mg NuFix® Glyoxal Agarose with a binding capacity of 0.280 meq/g (Cambrex Bio Science) in a 6.6 mL aqueous solution of 2.5 mM SHA-X-hydrazide (Cambrex Bio Science) and 8 mM acetic hydrazide (Sigma-Aldrich). The 1:3.2 ratio of SHA-X hydrazide to acetic hydrazide was assumed to be reflected in the corresponding immobilized groups. After 1 hour, the liquid was separated from the derivatized gel. The moist, derivatized gel was then divided. A major portion of the wet mass (454 mg) was suspended in 200 mL water and 5.85 g SeaKem LE Agarose added to produce sample F. The remaining minor portion of the west mass (45 mg) was suspended in 200 mL water and 5.985 g SeaKem LE Agarose added to produce sample G.

Each of the separate agarose suspensions was heated to boiling to dissolve the agarose. After cooling, the gels were cut into approximately 5 mm cubes, which were frozen and thawed, before drying in a convection oven. The resulting flakes were ground to pass a 1 mm mesh to yield SHA agarose derivatives as free flowing powders with identical SHA ligand concentrations.

### Example 3

### Preparation of Agarose Matrices with Varying Diffuse Ligand Concentration and Similar Gel Strength

A further pair of agarose samples (H and I) was prepared with varying ligand concentration and similar gel strengths. These differed from F and G in that the ratios of the SHA-derivatized NuFix® and the SeaKem® LE Agarose was kept constant, but the ratio of SHA Hydrazide to acetic hydrazide was varied to provide two levels of ligand spacing.

To prepare sample H, a derivatized agarose concentrate was prepared by suspending 1.5 g NuFix® Glyoxal Agarose with a binding capacity of 0.280 meq/g (Cambrex Bio Science) in a 60 mL aqueous solution of 0.25 mM SHA-X-hydrazide (Cambrex Bio Science) and 10.25 mM acetic hydrazide (Sigma-Aldrich). The 1:41 ratio of SHA-X-hydrazide to acetic hydrazide was assumed to be reflected in the corresponding immobilized groups. After 1 hour, the moist, derivatized gel suspension was further diluted with 140 mL water and 4.5 g SeaKem LE agarose added.

To prepare sample I, a derivatized agarose concentrate was prepared by suspending 1.5 g NuFix® Glyoxal Agarose with a binding capacity of 0.280 meq/g (Cambrex Bio Science) in a 60 mL aqueous solution of 0.025 mM SHA-X-hydrazide (Cambrex Bio Science) and 10.47 mM acetic hydrazide (Sigma-Aldrich). The 1:416 ratio of SHA-X-hydrazide to acetic hydrazide was assumed to be reflected in the corresponding immobilized groups. After 1 hour, the liquid was separated from the derivatized gel. The moist, derivatized gel suspension was further diluted with 140 mL water and 4.5 g SeaKem LE agarose added.

Each of the separate agarose suspensions was heated to boiling to dissolve the agarose. After cooling, the gels were cut into approximately 5 mm cubes, which were frozen and thawed, before drying in a convection oven. The resulting flakes were ground to pass a 1 mm mesh to yield SHA agarose derivatives as free flowing powders with identical SHA ligand concentration.

A summary of the different derivatives prepared (A-I) is given in Table 2.

TABLE 2
List of derivatives

| | | ligand | | SHA/AH ratio |
|---|---|---|---|---|
| | | concen- | weight fraction | on SHA-deriva- |
| | | tration | SHA-derivatized | tized polymer |
| ID | Sample | (μmol/g) | polymer | fraction |
| A | SeaKem Gold | 17 | 0.25 | 0.238 |
| B | SeaKem LE | 17 | 0.25 | 0.238 |
| C | SeaKem HGT | 17 | 0.25 | 0.238 |
| D | HSB-LV | 17 | 0.25 | 0.238 |
| E | SeaPlaque | 17 | 0.25 | 0.238 |
| F | SeaKem LE | 1.7 ^{a} | 0.025 | 0.238 |
| G | SeaKem LE | 0.17 ^{a} | 0.0025 | 0.238 |
| H | SeaKem LE | 1.7 ^{b} | 0.25 | 0.0238 |
| I | SeaKem LE | 0.17 ^{b} | 0.25 | 0.00238 |

^{a} clustered ligands
^{b} diffuse ligands

### Example 4

### Preparation of 25:1 PBA:Collagen

A solution of 4.79 mg/ml collagen (BD Biosciences) in 0.1 M sodium bicarbonate buffer, ph 8 was diluted with 0.1 M sodium bicarbonate, pH 8, to achieve a final concentration of 0.4 mg/ml collagen. A 100 mM solution of PBA reagent was prepared by dissolving 8 mg of PBA-X-NHS (Cambrex Bio Science) in 160 μl of anhydrous dimethylformamide. 1.7 μl of this PBA solution was pipetted directly into the collagen solution, the mixture vortexed for five seconds, and the reaction then cooled in the dark and on ice for one hour. The reaction mixture was purified by size exclusion by passing through a concentration determined by Bradford assay.

Results:

Tensile Strength Testing

To characterize physical properties of the agarose samples prepared above, each powdered sample was dissolved at 2.0% (w/w) in deionized water. Each agarose solution was poured between two glass plates a containing a 1.58 mm spacer and cooled to form a gel. The gel was removed from the glass plates and cut to size with a dumbbell-shaped cutter (DIN specification 53571). The gel samples were pulled at a rate of 50.00 mm/min in a test stand until fracture. The tensile strength and % elongation at fracture were recorded. The results of the tensile strength testing are given in Table 3.

TABLE 3
List of average agarose physical properties

| | | ligand | Tensile | |
|---|---|---|---|---|
| | | concentration | Strength | % Elongation |
| ID | Sample | (umol/g) | (N) | (%) |
| A | SeaKem Gold | 17 | 3.00 | 138 |
| B | SeaKem LE | 17 | 2.74 | 143 |
| C | SeaKem HGT | 17 | 1.64 | 131 |
| D | HSB-LV | 17 | 1.22 | 126 |
| E | SeaPlaque | 17 | 0.92 | 134 |
| F | SeaKem LE | 1.7 ^{a} | 2.31 | 136 |
| G | SeaKem LE | 0.17 ^{a} | 2.58 | 141 |
| H | SeaKem LE | 1.7 ^{b} | 2.21 | 137 |
| I | SeaKem LE | 0.17 ^{b} | 2.28 | 139 |

^{a} clustered ligands
^{b} diffuse ligands

Cell Culture Performance:

Five matrices described above (A-D) were evaluated as culture substrates. 1% agarose matrices were cast at 300 μL/well of a 24 well culture plate (BD Biosciences). Triplicate wells of each matrix were then exposed to cell adhesive biomolecules—Collagen I control (BD Biosciences) or PBA-linked Collagen I (25:1 ratio)—for 96 hours at 37° C. Fresh rat hepatocytes (Cambrex Bio Science) were subsequently seeded into each well at 25,000 viable cells/cm² in HCM culture medium (Cambrex Bio Science). Cells were evaluated after 24 hours for morphology using phase contrast microscopy (Nikon TE300 inverted microscope, 100× magnification) and for attachment via cellular ATP content.

Results clearly indicate a binary response to the matrices with PBA-linked Collagen I. Sample ID A and B support a spread, monolayer morphology, while Sample ID C, D and E support a spheroidal, 3D-aggregate morphology. These morphological traits are known to be associated with substrate compliance and/or adhesivity as described by Powers, et al., Biotechnol. Bioeng. 53: 415-426 (1997) and Semler, et al., Biotechnol. Bioeng. 69: 359-369 (2000). Rigid materials are expected to resist deformation by intercellular adhesive and contractile forces, thereby preventing significant cellular aggregation while promoting cell attachment and spreading. Relatively malleable or compliant gels would be unable to resist such cellular forces, and would therefore be dominated by the process of maximizing intercellular interactions, leading to spheroidal cellular aggregation.

The observed cellular behavior in this case clearly correlates with relative tensile strength and elongation percentage in these five materials: the two stronger materials lead to monolayers, the three weaker materials lead to spheroids. Control wells showed a complete lack of cell spreading in all cases, indicating the presence of insignificant adhesion. These results are independent of cell attachment. Single factor ANOVA followed by Tukey multiple comparison testing shows statistically indistinguishable cell attachment in samples A, B and E. Since ligand (and thus biomolecule) concentration is held constant in these experiments, these results suggest that the physical properties of each matrix dictate cellular and tissue morphology.

It is understood that the disclosed methods and materials are not limited to the particular methodology, protocols, and reagents described as these may vary. Unless defined otherwise, all technical and scientific terms used herein have the same meanings as commonly understood by one of skill in the art to which the disclosed invention belongs. Publications cited herein and the material for which they are cited are specifically incorporated by reference. Modifications and variations will be obvious to those skilled in the art and are intended to come within the scope of the appended claims.

We claim:

1. A composition for tissue engineering or repair, drug delivery, diagnostics, biosensors, or bioprocess engineering, the composition comprising
(a) a biocompatible matrix or implant; and
(b) one or more ligands suitable for complexing one or more bioactive molecules attached to the matrix or implant;
wherein the ligand concentration of the matrix can be controlled independently of the strength of the matrix.

2. The composition of claim 1 wherein the biocompatible matrix is selected from the group consisting of biodegradable materials, non-degradable materials, and combinations thereof.

3. The composition of claim 2 wherein the biodegradable material is selected from the group consisting of polyanhydrides, polyglycolic acid, polyhydroxy acids such as polylactic acid, polyglycolic acid, and polylactic acid-glycolic acid copolymers, polyorthoesters, polyhydroxybutyrate, polyphosphazenes, polypropylfumerate, biodegradable polyurethanes and combinations thereof.

4. The composition of claim 2 wherein the nondegradable material is selected from the group consisting of polystyrenes, polyethylene vinyl acetates, polypropylenes, polymethacrylates, polyacrylates, polyethylene vinyl acetates, oxides, glass, polysilicates, polycarbonates, polytetrafluoroethylene, fluorocarbons, nylon, silicon rubber, stainless steel alloys, titanium alloys and combinations thereof.

5. The composition of claim 2 wherein the material is a natural occurring material selected from the group consisting of collagen, polyamino acids, polysaccharides, hydroxyapatite, and combinations thereof.

6. The composition of claim 1 wherein the ligand suitable for complexing a bioactive molecule has the structure shown below: 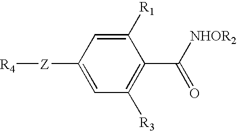
wherein
R₄ is a reactive electrophilic or nucleophilic moiety suitable for reaction of the phenyl boronic acid complexing reagent with the matrix material. R₂ is an H, an alkyl, or a methylene or ethylene moiety with an electronegative substituent. R₁ and R₃ are independently H or hydroxy and Z is optionally a spacer molecule comprising a saturated or unsaturated chain from 0 to 6 carbon equivalents in length, an unbranched saturated or unsaturated chain from 6 to 18 carbon equivalents in length with at least one intermediate amine or disulfide moieties, or a polyethylene glycol chain of 3 to 12 carbon equivalents in length.

7. The composition of claim 6 wherein the ligand suitable for complexing a bioactive molecule is salicylhydroxamine hydrazide.

8. The composition of claim 1 wherein the ligand suitable for complexing a bioactive molecule is streptavidin.

9. The composition of claim 1 wherein the ligand suitable for complexing a bioactive molecule is selected from the group consisting of aldehydes, activated esters, activated carboxylic acids, epoxides, and amines.

10. The composition of claim 1 wherein the complex formed is selected from the group consisting of irreversible covalent bonds, reversible covalent bonds, indirect conjugates, and combinations thereof.

11. The composition of claim 1 further comprising one or more therapeutic, diagnostic or prophylactic agents comprising one or more moieties complexed to the ligands.

12. The composition of claim 11 wherein the moiety is a phenyl boronic acid.

13. The composition of claim 11 wherein the moiety is biotin.

14. The composition of claim 11 wherein the moiety is selected from the group consisting of aldehydes, activated esters, activated carboxylic acids, epoxides, and amines.

15. The composition of claim 11 wherein the agents are therapeutics to be delivered to an intended site by release from the matrix or implant.

16. The composition of claim 11 wherein the one or more agents contains a spacer molecule between the one or more biomolecules and the one or more moieties in order to increase the mobility of the biomolecules in aqueous solution.

17. The composition of claim 16 wherein the spacer molecule comprises a spacer selected from the group consisting of aliphatic chains up to about 6 carbon equivalents in length, unbranched aliphatic chains of 6 to 18 carbon equivalents in length with at least one of an intermediate amide or disulfide moiety, or a polyethylene oxide or polyethylene glycol chain of 3-12 carbon equivalents in length.

18. The composition of claim 1 wherein multiple ligands are present, each capable of selectively immobilizing a bioreactive species by an interaction selected from the group consisting of irreversible covalent bonds, reversible covalent bonds, indirect conjugates, and combinations thereof.

19. The composition of claim 1 suitable for use in tissue repair or tissue engineering.

20. The composition of claim 1 suitable for use in cell culture.

21. The composition of claim 1 as a liquid or suspension for application to bone.

22. The composition of claim 1 wherein the concentration of the matrix material is independent of the ligand concentration.

23. The composition of claim 1 wherein the matrix comprises polymers or monomers having a defined ligand concentration and polymers or monomers that do not have ligands bound thereto, wherein the ligand concentration can be independently varied without altering the concentration of the polymer forming the matrix.

24. The composition of claim 1 comprising ligand or receptor covalently coupled to the matrix or implant, wherein the ligand or receptor can be released without proteolytic cleavage.

25. A method of making the composition of claim 1, the method comprising
(a) selecting a biocompatible matrix or implant for cell culture, tissue repair or engineering or drug delivery; and
(b) attaching to the matrix or implant one or more ligands suitable for complexing a therapeutic, prophylactic or bioactive molecules comprising one or more reactive moieties.

26. A method of making a hydrogel or organogel matrix for use as a tissue engineering matrix or cell culture substrate, having ligands bound thereto in a defined concentration, wherein the ligand concentration is obtained by mixing the hydrogel or organogel with ligands bound to the monomers or polymers forming the hydrogel or organogel with hydrogel or organogel not having ligands bound to the monomers or polymers forming the hydrogel or organogel.

27. A method of cell culture, drug delivery or tissue repair or engineering comprising providing the composition of claim 1 and adding cells or implanting the matrix or implant.
